(19) United States
(12) Patent Application Publication
(10) Pub. No.: US 2016/0175553 A1
(43) Pub. Date: Jun. 23, 2016

(54) LOW RESISTANCE AEROSOL EXHALATION FILTER

(71) Applicant: INSMED INCORPORATED, Bridgewater, NJ (US)

(72) Inventors: Walter R. PERKINS, Neshanic Station, NJ (US); Zhili LI, Kendall Park, NJ (US)

(21) Appl. No.: 14/905,274

(22) PCT Filed: Jul. 17, 2014
(86) PCT No.: PCT/US14/47010

§ 371 Date: Jan. 14, 2016

Related U.S. Application Data
(60) Provisional application No. 61/847,324, filed on Jul. 17, 2013.

(51) Int. Cl. A61M 16/10; A61M 15/00; A61K 47/24; A61K 9/127; A61K 9/00; A61K 47/28; A61M 11/00; A61K 31/7036

(52) U.S. Cl. CPC A61M 16/1065; A61M 11/00; A61M 15/0018; A61K 31/7036; A61K 9/127; A61K 9/0073; A61K 47/28; A61K 47/24

(57) ABSTRACT

Low resistance aerosol exhalation filters, and methods of their use are provided. The exhalation filters provided herein are used in conjunction with a nebulizer in the treatment of a pulmonary infection in a patient in need thereof. In one method, an antiinfective formulation is administered to a patient in need of treatment of a pulmonary infection, with a nebulizer comprising a low resistance aerosol exhalation filter of the invention. The pulmonary infection, in one embodiment, is a Pseudomonas or mycobacterial (e.g., NTM) infection.

### CROSS-REFERENCE TO RELATED APPLICATION

This application claims priority from U.S. Provisional Application Ser. No. 61/847,324, filed Jul. 17, 2013, which is hereby incorporated by reference in its entirety for all purposes.

### BACKGROUND OF THE INVENTION

Liposomal aminoglycoside formulations can be administered to patients for the treatment of chronic Pseudomonas aeruginosa infections in cystic fibrosis (CF) patients as well as chronic infections caused by non-tuberculous mycobacteria (NTM). Liposome encapsulation of aminoglycoside (e.g., amikacin) reduces non-specific binding of this cationic aminoglycoside drug to the negatively charged mucus and biofilm surfaces in CF patients and allows penetration and delivery of packets of highly concentrated drug to the otherwise protected bacteria; liposomal uptake into NTM infected alveolar macrophages also increases drug access to those organisms.

Many drugs have been delivered by nebulization. Patients typically inhale nebulized drug formulations, for example, Arikace®, a liposomal amikacin formulation, through a mouthpiece. The mouthpiece has an opening through which exhaled air passes but through which air cannot enter on inhalation, i.e., a one-way exhalation valve. The exhaled air enters the local environment around the patient. Most nebulizers have substantially the same basic scheme regarding exhaled air, although the specific configurations differ. In those instances where it was/is desirable to capture the exhaled aerosol, the usual exhalation pathway is altered so that air passes through a filter. The filter (replaceable filter pad) is contained in a housing or cartridge through which the air passes.

The surface area of all of these exhalation filters is fixed. For nebulization of small volumes, these filters likely capture exhaled aerosol adequately. However, as with any air filter, progressive wetting of the filter increases resistance to air flow. Air passage through a fully wet filter requires substantial pressure. For patients, this resistance and back pressure equates to more effort to exhale. This situation tires patients unnecessarily and may result in patients discontinuing their treatment prematurely and/or discontinuing use of the inhaled therapy altogether.

The present invention addresses this and other needs.

### SUMMARY OF THE INVENTION

In one aspect, a low resistance aerosol exhalation filter is provided. The exhalation filter is attached to a nebulizer for use during nebulization therapy, for example in the treatment of a pulmonary infection in a patient in need thereof. In one embodiment, the pulmonary infection is a Psuedomonas or a mycobacterial infection.

In another aspect, a method for treating a patient with a pulmonary infection is provided. In one embodiment, the method comprises administering a nebulized drug formulation to the patient in need of treatment, wherein the formulation is administered via a nebulizer system comprising the low resistance aerosol exhalation filter of the present invention. In a further embodiment, the drug formulation is a liposomal aminoglycoside formulation. In a further embodiment, the lipids in the liposomal formulation comprise a phosphatidylcholine and a sterol. In even a further embodiment, the pulmonary infection is a Pseudomonas infection or an NTM infection.

### BRIEF DESCRIPTION OF THE FIGURES

FIG. 1 is a diagram of a small volume jet nebulizer (single patient use-disposable) from Salter Labs® Filter Set-Disposable for NebuTech® HDN® Nebulizer (left); and a partial cross sectional view of a filter housing (adapted from U.S. Pat. No. 6,631,721) (right).

FIG. 2 is a schematic diagram illustrating nebulizer configurations for a two minute protocol (left, A) and a five breath dosimeter protocol (right, B). Both include an exhalation filter. (adapted from Am J Respir Crit Care Med. v. 161, pp. 309-329, 2000).

FIG. 3 is an image of a nebulizer filter/valve set, designed to filter a patient's exhalation breath during nebulization treatments.

FIG. 4 shows one embodiment of a low resistance exhalation filter of the present invention. Specifically, a ‘bag’ exhalation filter system for use with a nebulizer. The large surface area of the ‘bag’ filter provides low resistance to air flow.

FIG. 5 shows an example of a one-way plastic valve that could potentially be part of connector/bag to prevent passage of air into the inhalation air stream. (adapted from Biopac Systems, Inc. website).

FIG. 6 is an example of a one-way paper tube valve. The paper tube could potentially be an insert in the connector or ‘neck’ of the bag that provides valve mechanism.

### DETAILED DESCRIPTION OF THE INVENTION

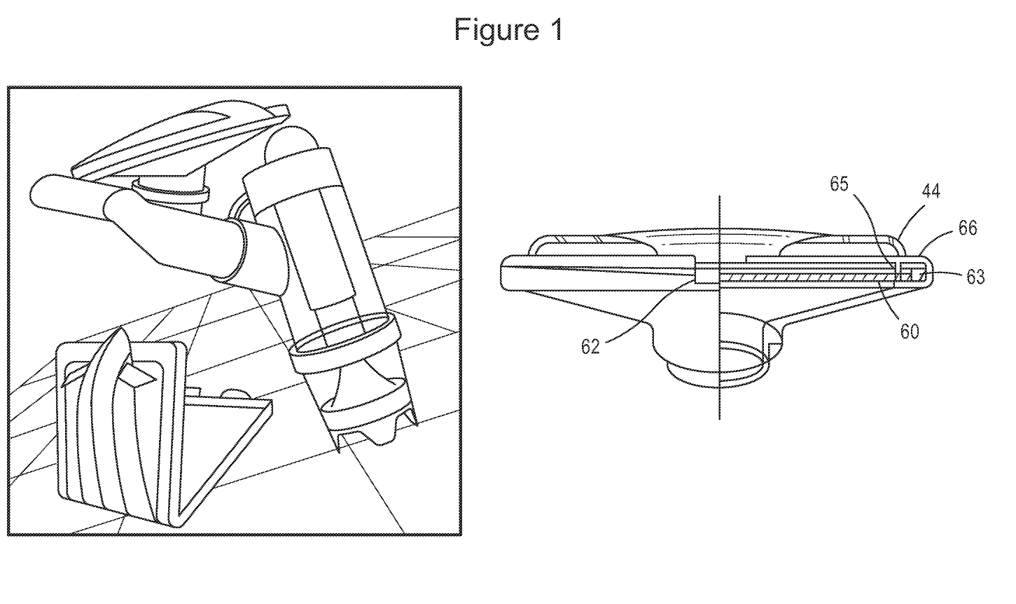

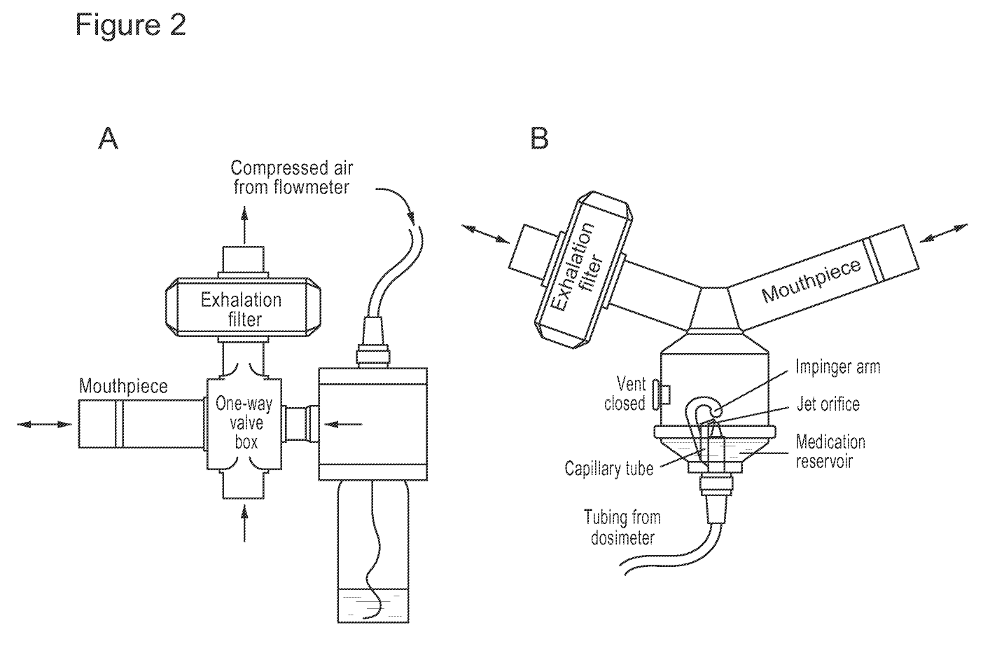

Examples of filters and the nebulizer configurations in which they are used are shown in FIGS. 1-3. Current exhalation filters such as those shown in FIGS. 1-3, suffer from limitations, making their use problematic for patients undergoing nebulization therapy. Problems with current systems include:
limited surface area which may lead to resistance in air flow due to wetting,
filter replacement during nebulization, and
burdensome cleaning and handling steps.

In contrast, an improved exhalation filter should include one or more of the following features:
have a low resistance to air flow,
not be affected by wetting of the filter,
be disposable to alleviate the need for cleaning a filter holder, and
be easy to attach and detach to the nebulizer.

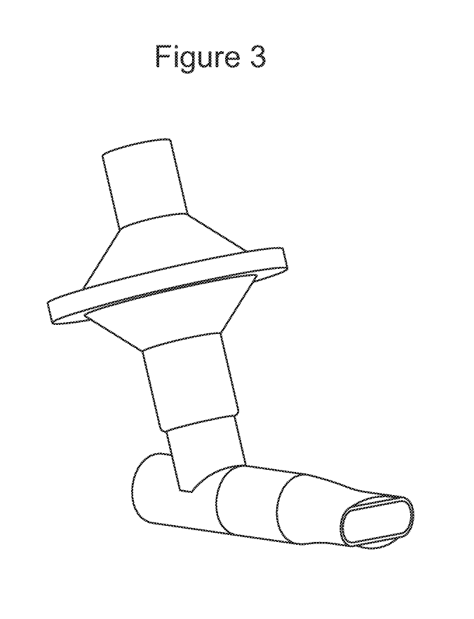

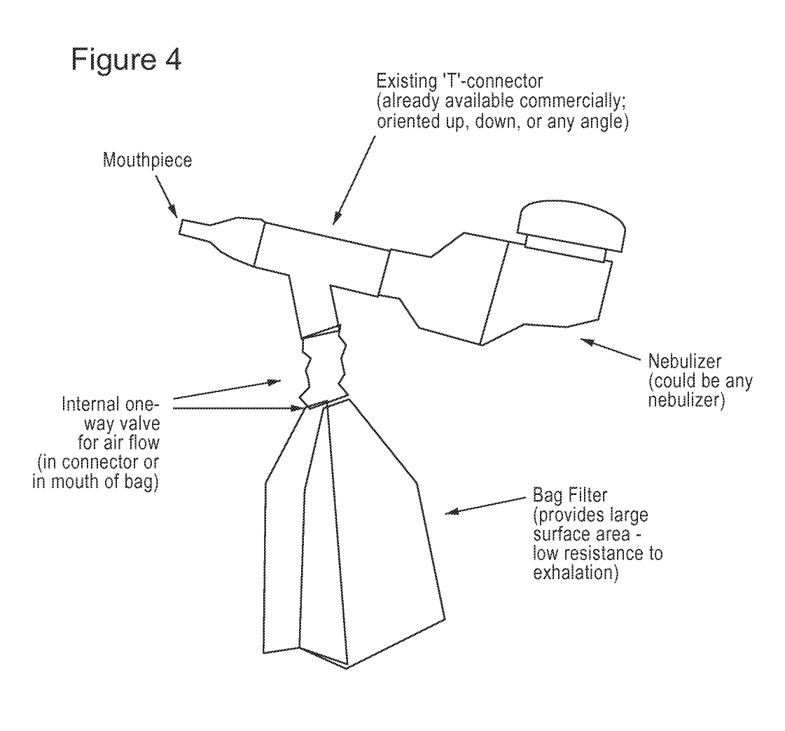

Embodiments of the low resistance nebulizer exhalation filter of the invention are provided in FIG. 4. The ‘T’ connector described herein is commercially available from different vendors as the connection is of a size that fits standard ventilator tubing. One ‘T’ connector according to an embodiment is provided in FIG. 3.

In one embodiment, the low resistance exhalation filter is comprised of paper. In another embodiment the low resistance exhalation filter is comprised of cloth. In yet another embodiment, the low resistance exhalation filter is comprised of fiber. In still another embodiment, the low resistance exhalation filter is comprised of plastic. The low resistance exhalation filter, in some embodiments, is constructed of two materials selected from cloth, fiber, plastic and paper. One of skill in the art will appreciate that the low resistance exhalation filter, regardless of material, should be a ‘breathable’ mesh through which air flows freely but aerosol droplets do not.

The low resistance exhalation filter is not limited by size and/or dimensions. The size and/or dimensions of the filter can be varied depending on the nebulizer that the filter is used with.

A tube connecting the body of the filter ‘bag’ to the ‘T’ (e.g., FIGS. 3 and 4), in one embodiment, is constructed from paper, cloth, fiber, plastic, or a combination thereof. In one embodiment, the connecting tube is physically attached to the filter bag. The shape of the opening of the connector is conducive to it being slipped over the open end of the ‘T’, such that a snug fit is attained where air cannot pass between the contact areas of the connecting tube and ‘T’. However, the fit should not be so tight that removal of the connector off of the ‘T’ is difficult.

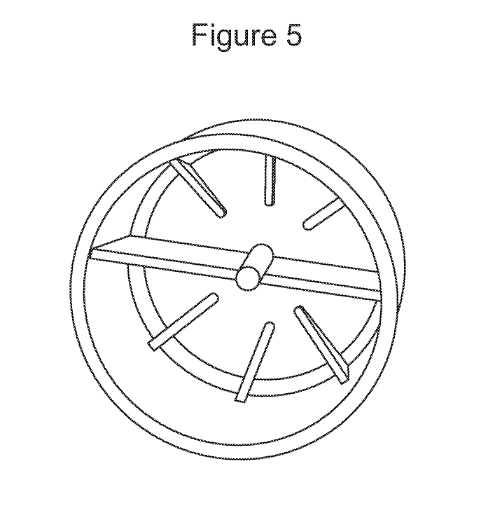

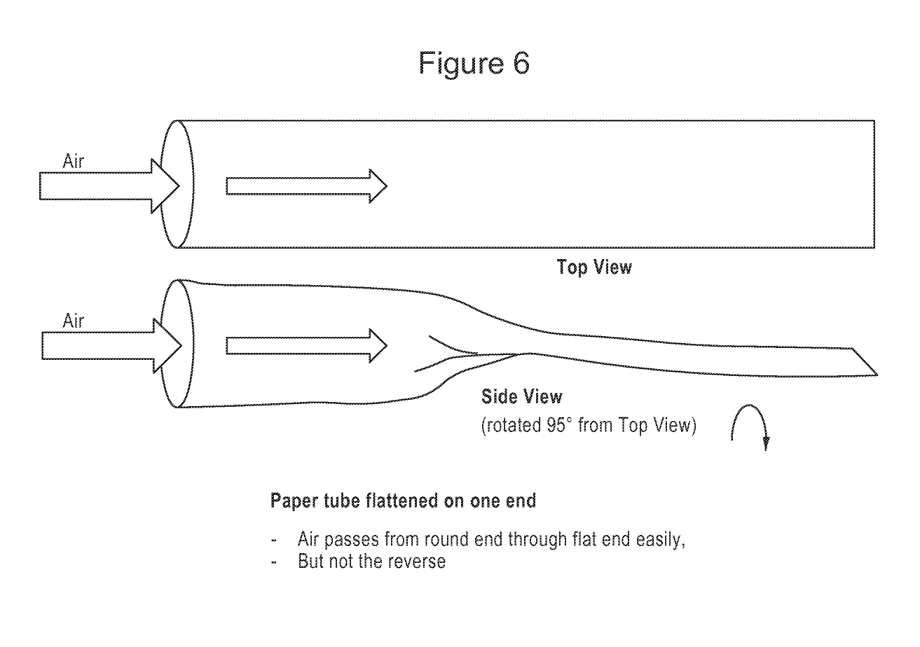

On exhalation, the patient's breath will pass through the ‘T’ and into the bag filter, where air passes out through the walls of the filter bag but aerosol is retained. On inhalation, there is no flow of air, or minimal flow of air, from the bag back to the patient. Only air originating from the nebulizer is inhaled when the aerosol filter of the present invention is attached to a nebulizer. To facilitate the air control, in one embodiment, there is a one-way valve situated in the connector, or at the point between the connector and bag where exhaled air enters the bag. Examples of one-way air valves are in FIGS. 5 and 6.

In FIG. 5, the one-way valve is made on plastic and is of the appropriate dimension to fit ventilator tubing. A plastic ‘flap’ in the valve affords unidirectional air flow.

In FIG. 6, a one-way valve is conceptualized as a tube that is collapsed flat at one end. Air passes from the open end of the tube through the flattened end freely while air does not flow in the reverse direction. This type of tube, in one embodiment, is inserted into the connector such that the flattened portion is inside the filter bag and the open end is aligned with the connector. This type one-way valve could be constructed of paper, plastic, a combination thereof, or other materials.

In one embodiment, all parts of the filter and nebulizer, except ‘T’, are to be disposed after each use. Therefore, materials and production must be cost effective.

The aerosol filters described herein are amenable for use with any type of nebulizer. For example, pneumonic (jet), vibrating mesh, ultrasonic, electronic nebulizers, e.g., passive electronic mesh nebulizers, active electronic mesh nebulizers and vibrating mesh nebulizers are amenable for use with the invention. In one embodiment, the filter provided herein is used in conjunction with a nebulizer selected from an electronic mesh nebulizer, pneumonic (jet) nebulizer, ultrasonic nebulizer, breath-enhanced nebulizer and breath-actuated nebulizer. In one embodiment, the nebulizer is portable. In another embodiment, the nebulizer is a single use, disposable nebulizer.

In one embodiment, the filter provided herein is used in conjunction with a continuous nebulizer. In other words, refilling the nebulizer with a pharmaceutical formulation while administering a dose is not needed. Rather, in one embodiment, the nebulizer has at least an 8 mL capacity or at least a 10 mL capacity. In one embodiment, the capacity of the nebulizer used in conjunction with the filter described herein is about 8 mL or about 10 mL.

In one embodiment, the low resistance one way filter provided herein is used in conjunction with a nebulizer to administer an antiinfective drug to a patient in need of treatment of a pulmonary infection. For example, in one embodiment, the antiinfective drug is an aminoglycoside. In a further embodiment, the aminoglycoside is selected from amikacin, apramycin, arbekacin, astromicin, capreomycin, dibekacin, framycetin, gentamicin, hygromycin B, isepamicin, kanamycin, neomycin, netilmicin, netilmicin, paromomycin, rhodestreptomycin, ribostamycin, sisomicin, spectinomycin, streptomycin, tobramycin or verdamicin. In one embodiment, the drug is amikacin (e.g., amikacin sulfate).

In one embodiment, at least one phospholipid is present in the pharmaceutical formulation. In one embodiment, the phospholipid is selected from: phosphatidylcholine (EPC), phosphatidylglycerol (PG), phosphatidylinositol (PI), phosphatidylserine (PS), phosphatidylethanolamine (PE), and phosphatidic acid (PA); the soya counterparts, soy phosphatidylcholine (SPC); SPG, SPS, SPI, SPE, and SPA; the hydrogenated egg and soya counterparts (e.g., HEPC, HSPC), phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The carbon chains on these fatty acids can be saturated or unsaturated, and the phospholipid may be made up of fatty acids of different chain lengths and different degrees of unsaturation.

In one embodiment, the pharmaceutical formulation includes dipalmitoylphosphatidylcholine (DPPC), a major constituent of naturally-occurring lung surfactant. In one embodiment, the lipid component of the pharmaceutical formulation comprises DPPC and cholesterol, or consists essentially of DPPC and cholesterol, or consists of DPPC and cholesterol. In a further embodiment, the DPPC and cholesterol have a mole ratio in the range of from about 19:1 to about 1:1, or about 9:1 to about 1:1, or about 4:1 to about 1:1, or about 2:1 to about 1:1, or about 1.86:1 to about 1:1. In even a further embodiment, the DPPC and cholesterol have a mole ratio of about 2:1 or about 1:1. In one embodiment, DPPC and cholesterol are provided in an aminoglycoside formulation, e.g., an aminoglycoside formulation.

Other examples of lipids for use with the invention include, but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE).

In one embodiment, the at least one lipid component comprises a sterol. In a further embodiment, the at least one lipid component comprises a sterol and a phospholipid, or consists essentially of a sterol and a phospholipid, or consists of a sterol and a phospholipid. Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

In one embodiment, at least one cationic lipid (positively charged lipid) is provided in the systems described herein. The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio) propane (DOTAP).

In one embodiment, at least one anionic lipid (negatively charged lipid) is provided in the systems described herein. The negatively-charged lipids which can be used include phosphatidylglycerols (PGs), phosphatidic acids (PAs), phosphatidylinositols (PIs) and the phosphatidyl serines (PSs). Examples include DMPG, DPPG, DSPG, DMPA, DPPA, DSPA, DMPI, DPPI, DSPI, DMPS, DPPS and DSPS.

Without wishing to be bound by theory, phosphatidylcholines, such as DPPC, aid in the uptake of the aminoglycoside agent by the cells in the lung (e.g., the alveolar macrophages) and helps to maintain the aminoglycoside agent in the lung. The negatively charged lipids such as the PGs, PAs, PSs and PIs, in addition to reducing particle aggregation, are thought to play a role in the sustained activity characteristics of the inhalation formulation as well as in the transport of the formulation across the lung (transcytosis) for systemic uptake. The sterol compounds, without wishing to be bound by theory, are thought to affect the release characteristics of the formulation.

Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (see, e.g., Cullis et al. (1987)). In one embodiment, one or more of the methods described in U.S. Patent Application Publication No. 2008/0089927 are used herein to produce the aminoglycoside encapsulated lipid formulations (liposomal dispersion). The disclosure of U.S. Patent Application Publication No. 2008/0089927 is incorporated by reference in its entirety for all purposes. For example, in one embodiment, at least one lipid and an aminoglycoside are mixed with a coacervate (i.e., a separate liquid phase) to form the liposome formulation. The coacervate can be formed to prior to mixing with the lipid, during mixing with the lipid or after mixing with the lipid. Additionally, the coacervate can be a coacervate of the active agent.

The lipid to drug weight ratio in the pharmaceutical formulations delivered via the low resistance filter provided herein, in one embodiment, is 3 to 1 or less, 2.5 to 1 or less, 2 to 1 or less, 1.5 to 1 or less, or 1 to 1 or less. The lipid to drug ratio in the pharmaceutical formulations provided herein, in another embodiment, is less than 3 to 1, less than 2.5 to 1, less than 2 to 1, less than 1.5 to 1, or less than 1 to 1. In a further embodiment, the lipid to drug weight ratio is about 0.7 to or less or about 0.7 to 1. The lipid to drug weight ratio in the pharmaceutical formulations delivered via the low resistance filter provided herein, in another embodiment, is from about 3:1 (lipid:drug) to about 0.25:1 (lipid:drug), or about 2.5:1 (lipid:drug) to about 0.50:1 (lipid:drug), or about 2.0:1 (lipid:drug) to about 0.5:1 (lipid:drug), or about 1.5:1 (lipid:drug) to about 0.5:1 (lipid:drug), or about 1:1 (lipid:drug) to about 0.5:1 (lipid:drug).

In one embodiment, the formulation delivered via the filters provided herein comprises an aminoglycoside, for example, amikacin, e.g., amikacin base. In one embodiment, the amount of aminoglycoside provided in the formulation is about 450 mg, about 500 mg, about 550 mg, about 560 mg, about 570 mg, about 580 mg, about 590 mg, about 600 mg or about 610 mg. In another embodiment, the amount of aminoglycoside is from about 500 mg to about 600 mg, or from about 500 mg to about 650 mg, or from about 525 mg to about 625 mg, or from about 550 mg to about 600 mg. In one embodiment, the aminoglycoside is provided in about an 8 mL formulation or about a 10 mL formulation, and is administered to a patient in need thereof in a once-daily dosing session. In a further embodiment, the formulation comprises about 560 mg to about 700 mg aminoglycoside, e.g., about 590 mg aminoglycoside.

In one embodiment, the pulmonary infection treated with the nebulizer and low resistance aerosol filter, and formulations described herein is a Pseudomonas (e.g., P. aeruginosa, P. paucimobilis, P. putida, P. fluorescens, and P. acidovorans), Burkholderia (e.g., B. pseudomallei, B. cepacia, B. cepacia complex, B. dolosa, B. fungorum, B. gladioli, B. multivorans, B. vietnamiensis, B. pseudomallei, B. ambifaria, B. andropogonis, B. anthina, B. brasilensis, B. caledonica, B. caribensis, B. caryophylli), Staphylococcus (e.g., S. aureus, S. auricularis, S. carnosus, S. epidermidis, S. lugdunensis), Methicillin-resistant Staphylococcus aureus (MRSA), Streptococcus (e.g., Streptococcus pneumoniae), Escherichia coli, Klebsiella, Enterobacter, Serratia, Haemophilus, Yersinia pestis, Mycobacterium (e.g., nontuberculous mycobacterium, M. abscessus, M. chelonae, M. bolletii, M. tuberculosis, M. avium complex (MAC) (M. avium and M. intracellulare), M. kansasii, M. xenopi, M. marinum, M. ulcerans, or M. fortuitum complex (M. fortuitum and M. chelonae)). In a further embodiment, the patient is a cystic fibrosis patient.

In another embodiment, the low resistance one way filter provided herein is used in conjunction with a nebulizer to administer an antiinfective drug to a patient in need of treatment of a nontuberculous mycobacterial (NTM) infection. In a further embodiment, the NTM infection is M. abscessus, M. chelonae, M. bolletii, M. kansasii, M. simiae, M. ulcerans, M. avium (e.g., M. avium subsp. hominissuis), M. avium complex (MAC) (M. avium and M. intracellulare), M. kansasii, M. peregrinum, M. xenopi, M. marinum, M. malmoense, M. terrae, M. haemophilum, M. genavense, M. ulcerans, M. fortuitum or M. fortuitum complex (M. fortuitum and M. chelonae). As provided above, the antiinfective drug, in one embodiment is in a liposomal formulation. In a further embodiment, the lipids in the liposomal formulation comprise a phospholipid and a sterol. In even a further embodiment, the phospholipid is DPPC and cholesterol.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A low resistance aerosol exhalation filter comprising,
means for controlling air flow upon patient exhalation.

2. The low resistance aerosol exhalation filter of claim 1, wherein the means for controlling air flow comprises a one-way valve.

3. The low resistance aerosol exhalation filter of claim 1 or 2, wherein the filter is comprised of cloth, plastic, fiber, paper, or a combination thereof.

4. A nebulizer comprising the low resistance aerosol exhalation filter of any one of claims 1-3.

5. The nebulizer of claim 4, wherein the nebulizer is single use and disposable.

6. A method for treating a pulmonary infection in a patient in need thereof, comprising,
administering a nebulized drug formulation to the patient in need of treatment with the nebulizer of claim 4 or 5.

7. The method of claim 6, wherein the drug formulation comprises an aminoglycoside.

8. The method of claim 7, wherein the aminoglycoside is amikacin, apramycin, arbekacin, astromicin, capreomycin, dibekacin, framycetin, gentamicin, hygromycin B, isepamicin, kanamycin, neomycin, netilmicin, netilmicin, paromomycin, rhodestreptomycin, ribostamycin, sisomicin, spectinomycin, streptomycin, tobramycin or verdamicin.

9. The method of claim 7, wherein the aminoglycoside is amikacin.

10. The method of claim 9, wherein the amikacin is amikacin sulfate.

11. The method of any one of claims 6-10, wherein the drug formulation is a liposomal drug formulation.

12. The method of claim 11, wherein the lipid in the liposomal formulation comprises a phospholipid and a sterol.

13. The method of claim 12, wherein the sterol is cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherol, tocopherol hemi-succinate, tocopherol hydrogen sulfate or tocopherol sulfate.

14. The method of claim 12, wherein the sterol is cholesterol.

15. The method of any one of claims 12-14, wherein the phospholipid is a phosphatidylcholine, phosphatidylglycerol, phosphatidylinositol, phosphatidylserine, phosphatidylethanolamine or phosphatidic acid.

16. The method of any one of claims 12-14, wherein the phospholipid is dipalmitoylphosphatidylcholine (DPPC), dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), or mono-oleoyl-phosphatidylethanolamine (MOPE)

17. The method of any one of claims 12-14, wherein the phospholipid is a phosphatidylcholine.

18. The method of claim 17, wherein the phosphatidylcholine is dipalmitoylphosphatidylcholine (DPPC).

19. The method of any one of claims 11-18, wherein the lipid to drug weight ratio of the formulation is less than 3 to 1, less than 2.5 to 1, less than 2 to 1, less than 1.5 to 1, or less than 1 to 1.

20. The method of claim 19, wherein the lipid to drug weight ratio is about 0.7 to 1 or less or about 0.7 to 1.

21. The method of any one of claims 11-18, wherein the lipid to drug weight ratio of formulation is from about 3:1 (lipid:drug) to about 0.25:1 (lipid:drug), or about 2.5:1 (lipid:drug) to about 0.50:1 (lipid:drug), or about 2.0:1 (lipid:drug) to about 0.5:1 (lipid:drug), or about 1.5:1 (lipid:drug) to about 0.5:1 (lipid:drug), or about 1:1 (lipid:drug) to about 0.5:1 (lipid:drug).

22. The method of any one of claims 6-21, wherein the pulmonary infection is a Pseudomonas infection.

23. The method of any one of claims 6-21, wherein the pulmonary infection is a mycobacterial infection.

24. The method of claim 22, wherein the Pseudomonas infection is a Pseudomonas aeruginosa infection.

25. The method of claim 23, wherein the mycobacterial infection is a nontuberculous mycobacterial (NTM) infection.

26. The method of claim 25, wherein the NTM infection is M. abscessus, M. chelonae, M. bolletii, M. kansasii, M. simiae, M. ulcerans, M. avium, M. avium complex (MAC) (M. avium and M. intracellulare), M. kansasii, M. peregrinum, M. xenopi, M. marinum, M. malmoense, M. terrae, M. haemophilum, M. genavense, M. ulcerans, M. fortuitum or M. fortuitum complex (M. fortuitum and M. chelonae).

27. The method of claim 26, wherein the M. avium infection is M. avium subsp. hominissuis.

28. The method of claim 26, wherein the NTM infection is M. abscessus.

29. The method of claim 26, wherein the NTM infection is M. chelonae.
